(19) United States
(12) Patent Application Publication
(10) Pub. No.: US 2014/0058434 A1
(43) Pub. Date: Feb. 27, 2014

(54) RELEASABLE DEVICE SYSTEM

(71) Applicants: Donald K. Jones, Dripping Springs, TX (US); Vladimir Mitelberg, Austin, TX (US)

(72) Inventors: Donald K. Jones, Dripping Springs, TX (US); Vladimir Mitelberg, Austin, TX (US)

(21) Appl. No.: 13/971,817

(22) Filed: Aug. 20, 2013

Related U.S. Application Data
(60) Provisional application No. 61/691,464, filed on Aug. 21, 2012.

(51) Int. Cl. A61B 17/12; A61B 17/34

(52) U.S. Cl. CPC A61B 17/1214; A61B 17/3468
USPC 606/200

(57) ABSTRACT

A medical implant deployment system for placing an implant at a preselected site within a vessel, duct or body lumen of a mammal. The reusable deployment system includes a mechanical coupling assembly at the distal end of a positioning member, having a first configuration in which the coupling assembly distal end is insertable or removable from the implant proximal end and a second configuration where the coupling assembly distal end is interlockingly engaged with the implant proximal end. Once the implant is properly positioned the coupling assembly is actuated, thereby releasing the implant at a desired position within the body.

### BACKGROUND OF THE INVENTION

For many years flexible catheters have been used to place various devices within the vessels of the human body. Such devices include dilatation balloons, radio-opaque fluids, liquid medications and various types of occlusion devices such as balloons and embolic coils. Examples of such catheter devices are disclosed in U.S. Pat. No. 5,108,407, entitled “Method And Apparatus For Placement Of An Embolic Coil”; U.S. Pat. No. 5,122,136, entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils to preselected positions within vessels of the human body in order to treat aneurysms, or alternatively, to occlude the blood vessel at the particular location.

Coils which are placed in vessels may take the form of helically wound coils, or alternatively, may be random wound coils, coils wound within other coils or many other such configurations. Examples of various coil configurations are disclosed in U.S. Pat. No. 5,334,210, entitled, “Vascular Occlusion Assembly; U.S. Pat. No. 5,382,259, entitled, “Vasoocclusion Coil With Attached Tubular Woven Or Braided Fibrous Coverings.” Embolic coils are generally formed of radiopaque metallic materials, such as platinum, gold, tungsten, or alloys of these metals. Often times, several coils are placed at a given location in order to occlude the flow of blood through the vessel by promoting thrombus formation at the particular location.

In the past, embolic coils have been placed within the distal end of the catheter. When the distal end of the catheter is properly positioned the coil may then be pushed out of the end of the catheter with, for example, a guidewire to release the coil at the desired location. This procedure of placement of the embolic coil is conducted under fluoroscopic visualization such that the movement of the coil through the vasculature of the body may be monitored and the coil may be placed at the desired location. With these placements systems there is very little control over the exact placement of the coil since the coil may be ejected to a position some distance beyond the end of the catheter.

Numerous procedures have been developed to enable more accurate positioning of coils within a vessel. Still another such procedure involves the use of a glue, or solder, for attaching the embolic coil to a guidewire which, is in turn, placed within a flexible catheter for positioning the coil within the vessel at a preselected position. Once the coil is at the desired position, the coil is restrained by the catheter and the guidewire is pulled from the proximal end of the catheter to thereby cause the coil to become detached from the guidewire and released from the catheter system. Such a coil positioning system is disclosed in U.S. Pat. No. 5,263,964, entitled, “Coaxial Traction Detachment Apparatus And Method.”

Another coil positioning system utilizes a catheter having a socket at the distal end of the catheter for retaining a ball which is bonded to the proximal end of the coil. The ball, which is larger in diameter than the outside diameter of the coil, is placed in a socket within the lumen at the distal end of the catheter and the catheter is then moved into a vessel in order to place the coil at a desired position. Once the position is reached, a pusher wire with a piston at the end thereof is pushed distally from the proximal end of the catheter to thereby push the ball out of the socket in order to release the coil at the desired position. Such a system is disclosed in U.S. Pat. No. 5,350,397, entitled, “Axially Detachable Embolic Coil Assembly.” One problem with this type of coil placement system which utilizes a pusher wire which extends through the entire length of the catheter and which is sufficiently stiff to push an attachment ball out of engagement with the socket at the distal end of the catheter is that the pusher wire inherently causes the catheter to be very stiff with the result that it is very difficult to guide the catheter through the vasculature of the body.

Yet another coil deployment system is disclosed in U.S. Pat. No. 5,261,916, entitled, “Detachable Pusher-Vasooclusive Coil Assembly with Interlocking Ball and Keyway Coupling.” This system includes a pusher member with a tubular portion at its distal end that has a keyway for receiving the enlarged bead of an embolic coil through the outer wall and into the lumen of the tubular portion. The enlarged bead of the coil is positioned within the keyway and a resilient wire coupling the bead to the coil extends axially over the outer diameter of the distal end of the tubular portion to the remaining portion of the coil. The enlarged bead is retained in the keyway, forming an interlocking arrangement, by positioning the assembly within the lumen of an outer sleeve. Once the keyway is pushed from the confines of the sleeve the bead can disengage from the keyway. With this system the inner diameter has to be sufficiently large to accommodate the stack up of the wire coupled to the bead and the diameter of the tubular portion. Also when placing coils in an aneurysm “packed” with coils, there may not be enough room for the enlarged bead to disengage from the keyway.

Another coil release system is disclosed in U.S. Pat. No. 5,895,391 to Farnholtz, entitled, “Ball Lock Joint and Introducer for Vaso-occlusive Member”. This system incorporates a tubular member having a portion of the wall cut away to receive at least a portion of an enlarged bead coupled to the proximal end of the embolic coil. A wire is placed within the lumen of the tubular member and cooperates to form an interference fit between the wire, bead and cut-away wall portion. To release the coil, the wire is pulled from the proximal end of the system to remove the interference fit with the bead and cut-away wall portion.

Still another coil deployment system utilizes a pair of jaws placed on the distal end of a pusher wire to position and release a coil. One such system is described in U.S. Pat. Nos. 5,601,600 and 5,746,769 to Ton et al., entitled, “Endoluminal Coil Delivery System Having A Mechanical Release Mechanism.” Ton discloses an elongate pusher wire having jaws at the distal end. The jaws include tip projections which are perpendicular to the longitudinal axis of the pusher wire and when positioned with the lumen of a collar fixed to the proximal end of a coil, interlockingly engage with matching detents placed in the wall of the collar. A tubular body is used to slide over the pusher wire to collapse the jaws and release the collar. The disclosed interlocking engagement between the jaws and collar prevents forward and backwards axial movement of the jaws relative to the collar and allows any torqueing force applied to the jaws to be translated to the collar and affixed coil. Transmission of torque from a coil delivery system to a coil during the treatment of aneurysm may be detrimental to precise placement of the coil. The coils may coils store the torque energy and upon release from the delivery system, release the stored energy causing the coils to move unpredictably. Ton also states that jaws may be fixed to the coil, but does not provide or disclose any information as to how this may be accomplished.

Another method for placing an embolic coil is that of utilizing a heat releasable adhesive bond for retaining the coil at the distal end of the catheter. One such system uses laser energy which is transmitted through a fiber optic cable in order to apply heat to the adhesive bond in order to release the coil from the end of the catheter. Such a method is disclosed in U.S. Pat. No. 5,108,407, entitled, “Method And Apparatus For Placement Of An Embolic Coil.” Such a system also suffers from the problem of having a separate, relatively stiff element which extends throughout the length of the catheter with resulting stiffness of the catheter.

Another method for placing an embolic coil is that of utilizing a heat responsive coupling member which bonds the coil to the distal end of a delivery system. One such system uses electrical energy which is transmitted through electrical conductors to create heat which is applied to the coupling member to thereby soften and yield the coupling member in order to release the coil from the end of the delivery system. Such a method is disclosed in U.S. Pat. No. 7,179,276, entitled, “Heated Vascular Occlusion Coil Deployment System.” Such a system suffers from the problem of having to pull an engagement member once the coupling is softened in order to release the coil.

### SUMMARY OF THE INVENTION

The present invention is directed toward a medical implant deployment system for use in placing a medical implant at a preselected site within the body of a mammal which includes an elongate delivery system having a coupling assembly at its distal end that releasably engages the proximal end of a medical implant. The delivery system includes an elongate tubular positioning member having proximal and distal ends. A coupling assembly is positioned at the distal end of the positioning member and includes a tubular shaft member having proximal and distal ends and an elongate flexible actuator member positioned within the lumen of the shaft member. The actuator member further includes a tip member fixedly secured to its distal end. The coupling assembly is releasably engaged with in a lumen of the medical implant at its proximal end. The coupling assembly has a retracted configuration in which the actuator tip member and the shaft member distal end cooperatively engage the proximal end of the medical implant to restrict distal movement of the implant relative to the coupling assembly. The coupling assembly also has an extended configuration wherein the actuator tip member extends distal to the shaft member distal end and both the shaft member distal end and actuator tip member are insertable into or removable from the lumen of the medical implant at its proximal end.

The delivery system along with the distally located and releasably coupled medical implant are slidably positioned within the lumen of a catheter whose distal end is positioned adjacent a target implantation site. The delivery system is advanced such that the implant proximal end and coupling assembly distal end exit the lumen of the catheter. Once the implant is in the desired location, the coupling assembly is moved from its retracted configuration to its extended configuration in which the actuator member is advanced distally, relative to the shaft member, causing the actuator tip member to move distally from the shaft member distal end, thereby removing the cooperative engagement of the actuator tip member and shaft member distal end that previously restricted distal movement of the implant. While in the extended configuration the coupling assembly is moved proximally to remove the shaft member distal end and actuator tip member from the lumen of the medical implant, thereby releasing the implant at the target site.

The tubular positioning and shaft members are formed utilizing construction techniques well known in the formation of catheters or microcatheters. These construction techniques include for example braiding, coiling, extruding, laser cutting, joining, laminating, fusing and welding of components or portions of components to provide a tubular member having sufficient pushability and flexibility to traverse the luminal tortuosity when accessing an intended implantation site.

In accordance with an aspect of the present invention, there is provided a medical implant that takes the form of an embolization device such as an embolic or vaso-occlusive coil for selective placement within a vessel, aneurysm, duct or other body lumen. Embolic coils are typically formed through the helical winding of a filament or wire to form an elongate primary coil. The wire or filament is typically a biocompatible material suitable for implantation and includes metals such as platinum, platinum alloys, stainless steel, nitinol and gold. Other biocompatible materials such as plastics groups including nylons, polyesters, polyolefins and fluoro-polymers may be processed to produce suitable filaments for forming coils. The wire usually has a circular cross-section, however, non-circular cross-sections, such as “D” shapes, are used in commercially available coils. The diameter of the wire may range from 0.0001″ to about 0.010″ and is largely dependent upon the particular clinical application for the coil. The diameter of the primary coil is generally dependent upon the wire diameter and the diameter of the mandrel used for winding. The primary coil diameter typically ranges from 0.002″ to about 0.060″ and is also dependent upon on the clinical application. The wound primary coil is typically removed from the mandrel leaving the coil with a lumen. In addition to the aforementioned method of winding a coil, there are other “mandrel-less” forming processes that are suitable for making primary coils that plastically deform the wire into coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil has a range from about 0.1 cm to about 150 cm with a preferred range of about 0.5 cm to about 70 cm. The distal end of the coil is typically rounded or beaded to make the coil end more atraumatic. Other variations of embolic coils suitable for use include stretch resistant coils, coils that incorporate a stretch resistant member(s) (within the coil lumen or exterior to the coil) that limits undesirable elongation of the primary coil during device manipulation and coated or modified coils that enhance occlusion through coils surface modifications, addition of therapeutics or volume filling materials (foams, hydrogels, etc.).

In accordance with another aspect of the present invention, the proximal end of the implant may include a generally tubular headpiece having an interior lumen. The headpiece is fixedly coupled to the implant and the lumen may optionally include a plurality of protrusions and or recesses that are adapted to engage the actuator tip member and shaft member distal end when the coupling assembly of the delivery system is in the retracted configuration.

In accordance with yet another aspect of the present invention, the proximal end of the headpiece may include a proximal end wall having an aperture that is contiguous with the lumen of the headpiece or a lumen of the implant. The proximal end of the headpiece may take the form of a flange. The actuator tip member and shaft member distal end of the delivery system are positioned through the end wall aperture and are adapted to cooperatively engage with the interior of the end wall when the coupling assembly is in the retracted configuration.

In accordance with still yet another aspect of the present invention there is provided a method of delivering an implant at a target site that includes: providing a delivery system having a coupling assembly; providing a medical implant having a proximal end adapted to engage the distal end of the delivery system; verifying that the coupling assembly of the delivery system is placed in an extended configuration; inserting the distal end of the coupling assembly within a lumen of the medical implant at its proximal end; operating the delivery system to place the coupling assembly in a retracted configuration; verifying that the delivery system appropriately engages the implant; positioning the medical implant and delivery system within the lumen of a catheter having a distal end adjacent to a target implant site; advancing the delivery system through the catheter such that the implant exits the catheter lumen at its distal end; positioning the implant in a desired location; operating the delivery system to place the coupling assembly in an extended configuration; removing the coupling assembly distal end from within the lumen of the medical implant and removing the delivery system from the catheter lumen.

In accordance with another aspect of the present invention there is provided a method for delivering additional implants using the same delivery system that further includes: providing an additional medical implant having a proximal end adapted to engage the distal end of the delivery system; verifying that the coupling assembly of the delivery system is placed in an extended configuration; inserting the distal end of the coupling assembly within a lumen of the additional medical implant at its proximal end; operating the delivery system to place the coupling assembly in a retracted configuration; verifying that the delivery system appropriately engages the additional implant; positioning the additional medical implant and delivery system within the lumen of a catheter having a distal end adjacent to a target implant site; advancing the delivery system through the catheter such that the additional implant exits the catheter lumen at its distal end; positioning the additional implant in a desired location; operating the delivery system to place the coupling assembly in an extended configuration; removing the coupling assembly distal end from within the lumen of the additional medical implant and removing the delivery system from the catheter lumen.

These aspects of the invention and the advantages thereof will be more clearly understood from the following description and drawings of a preferred embodiment of the present invention:

### BRIEF DESCRIPTION OF THE DRAWINGS

FIG. 1 is a partially sectioned view of a medical implant deployment system according to an embodiment of the present invention.

FIG. 2 is an enlarged partially sectioned view showing a distal portion of the medical implant deployment system of FIG. 1.

FIG. 3A is a partial perspective view of the delivery system distal end in a retracted configuration.

FIG. 3B is a partial perspective view of the delivery system distal end in an extended configuration.

FIG. 4 is a partially sectioned view of the delivery system distal end in a retracted configuration engaged with a headpiece affixed to an implant according to another embodiment of the medical implant deployment system.

FIG. 5 is a partially sectioned view of the delivery system distal end in a retracted configuration in relation to a partially sectioned coil.

FIG. 6 is a partially sectioned view of the delivery system distal end in an extended configuration in relation to a partially sectioned coil.

FIG. 7 is a partially sectioned view of the delivery system distal end in an extended configuration positioned within the lumen of a coil.

FIG. 8 is a partially sectioned view of the delivery system distal end in a retracted configuration positioned within the lumen of a coil.

FIG. 9 is a partially sectioned view of a medical implant deployment system according to another embodiment of the present invention.

FIG. 10 is an enlarged partially sectioned view showing a distal portion of the medical implant deployment system of FIG. 9.

FIG. 11A is a partial perspective view according to another embodiment of a delivery system distal end in a retracted configuration.

FIG. 11B is a partial perspective view according to another embodiment of a delivery system distal end in an extended configuration.

FIG. 12 is a partially sectioned view of the delivery system distal end in an extended configuration in relation to a partially sectioned coil.

FIG. 13 is a partially sectioned view of the delivery system distal end in an extended configuration positioned within the lumen of a coil.

FIG. 14 is a partially sectioned view of the delivery system distal end in a retracted configuration positioned within the lumen of a coil.

FIG. 15 is a partially sectioned view of the delivery system distal end in a retracted configuration positioned within the lumen of a coil and the positioning member engaging the coil.

FIG. 16 is a partially sectioned view of the delivery system distal end in an extended configuration positioned within the lumen of a coil and the positioning member engaging the coil.

FIG. 17 is a partially sectioned view of the delivery system distal end in an extended configuration positioned within the lumen of the positioning member.

### DETAILED DESCRIPTION OF THE INVENTION

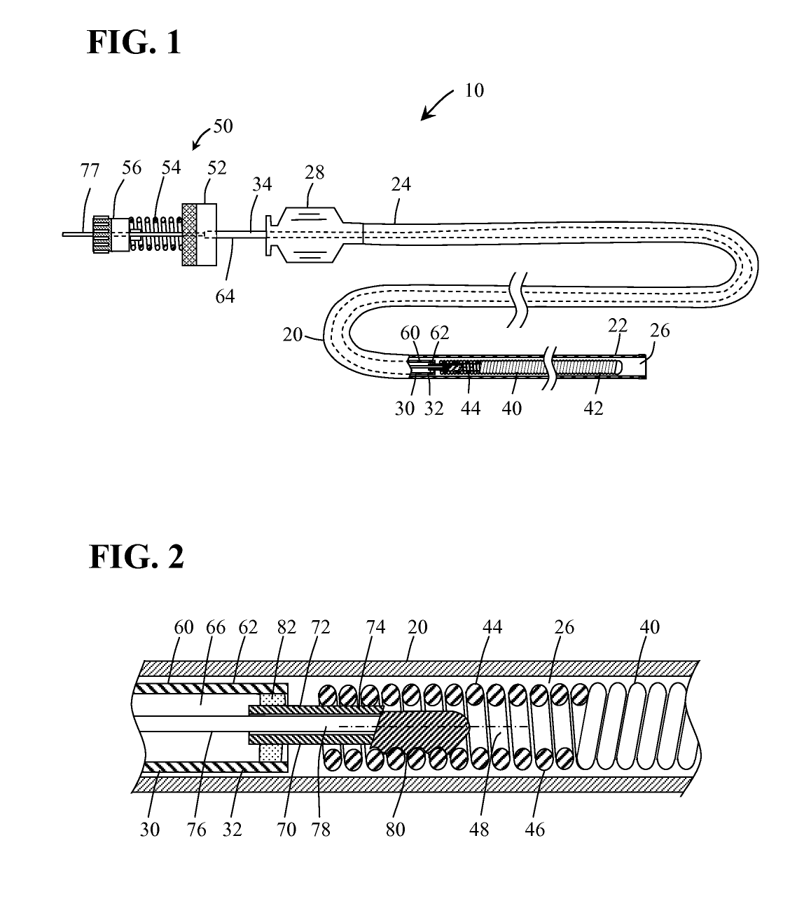

Generally, a medical implant deployment system of the present invention may be used to position an implant at a preselected site within the body of a mammal. The medical implant deployment system may be used to place various implants such as stents, filters, vascular occlusion devices, vascular plugs, aneurysm embolization devices and embolization coils. FIG. 1 generally illustrates a medical implant deployment system 10 of the present invention which includes delivery catheter 20 having a distal end 22, a proximal end 24, a lumen 26 extending therethrough and a catheter hub 28 affixed to proximal end 24, a delivery system 30 having a distal end 32 and a proximal end 34 and an embolic coil 40 having a distal end 42 and a proximal end 44 that is releasably coupled to the distal end 32 of delivery system 30. Embolic coil 40 is a medical implant of a general type suitable for use in occluding a vessel, duct or aneurysm.

Embolic coil 40 is generally formed from a primary coil of a helically wound wire 46, made from a material which is biocompatible and preferably radio-opaque. Suitable biocompatible materials include metals such as platinum, platinum alloys, stainless steel, nitinol, tantalum and gold and plastics such as nylons, polyesters, polyolefins and fluoropolymers. The wire usually has a circular cross-section, however, non-circular cross-sections, such as “D” shapes, are used in commercially available coils. The diameter of the wire may range from about 0.0001″ to about 0.010″ and is largely dependent upon the particular clinical application for the coil. The diameter of the primary coil is generally dependent upon the wire diameter and the diameter of the mandrel used for winding. The primary coil diameter typically ranges from about 0.002″ to about 0.060″ and is also dependent upon on the clinical application. The wound primary coil is typically removed from the mandrel leaving the coil with a lumen 48. In addition to the aforementioned method of winding a coil, there are other “mandrel-less” forming processes that are suitable for making primary coils that plastically deform the wire into coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil range from 0.1 cm to about 150 cm with a preferred range of about 0.5 cm to about 70 cm. The distal end of the coil is typically rounded or beaded to make the coil end more atraumatic. Other variations of embolic coils suitable for use include stretch resistant coils, coils that incorporate a stretch resistant member(s) (within the coil lumen or exterior to the coil) that limits undesirable elongation of the primary coil during device manipulation and coated or modified coils that enhance occlusion through coils surface modifications, addition of therapeutics or volume filling materials (foams, hydrogels, etc.).

As depicted in FIG. 1, deployment system 10 may further include an actuator assembly 50 that is positioned proximal to proximal end 24 of catheter 20. Actuator assembly 50 includes a first coupler member 52, a spacing member 54 and a second coupler member 56. The first and second coupler members 52 and 56 typically take the form of commercially available rotating hemostatic valve (RHV) like assemblies. A typical RHV-like assembly includes a housing body, a threaded cap and a compressible insert. The housing body and threaded cap are typically formed of a rigid plastic such as polystyrene, ABS, nylon or polycarbonate while the insert is formed of an elastomeric material such as silicone or rubber. The assembled housing body, cap and insert all have a contiguous aligned axial passage way. As the cap is threaded onto the housing body, the insert is compressed causing the diameter of the passageway through the insert to decrease. Spacing member 54 preferably takes the form of a spring and is positioned between the first coupler member 52 and the second coupler member 56. The ends of spacing member 54 may be releasably secured (or fixedly attached) to each of the coupler members.

FIG. 2 illustrates in more detail the construction of the implant deployment system 10 with the implant, coil 40, being positioned within catheter lumen 26 at catheter distal end 22. Delivery system 30 includes a tubular positioning member 60 having a distal end 62, a proximal end 64 and a lumen 66 extending therethrough. Delivery system 30 also includes a coupling assembly 70 having a tubular shaft member 72 with a distal end 74, and an elongate actuator member 76 with proximal and distal ends 77 and 78 respectively. Actuator member 76 preferably takes the form of an elongate resilient nitinol wire although other materials and forms such as tubes or cables may be suitable. Elongate actuator member 76 is positioned within the lumen of shaft member 72 and includes an actuator tip member 80 fixedly attached to actuator member distal end 78. Actuator tip member 80 is generally dimensioned to be greater than the lumen diameter of shaft member 72 at distal end 74 thereby preventing proximal movement of tip member 80 into the lumen of shaft member 72 at distal end 74. Shaft member 72 is shown positioned within lumen 66 and secured to positioning member 60 by securing means 82. Securing means 82 preferably takes the form of a UV curable adhesive but may take the form of any suitable joining technique such as soldering, laser welding and ultrasonic welding to bond shaft member 72 to distal end 62 of positioning member 60.

As previously discussed, the proximal end 44 of embolic coil 40 is releasably coupled to the distal end 32 of delivery system 30. More particularly, the distal end 74 of shaft member 72 and the actuator tip member 80 of coupling assembly 70 are positioned within lumen 48 of embolic coil 40 at proximal end 44. Shown in FIG. 2, delivery system 30 is in a retracted configuration where actuator tip member 80 is positioned adjacent shaft member distal end 74 such that the diameters of shaft member distal end 74 and actuator tip member 80 cooperatively engage the proximal end 44 of embolic coil 40 to thereby maintain the attachment of coupling assembly 70 to embolic coil 40. The actuator assembly 50 coupled to proximal end 34 of delivery system 30, operatively maintains the delivery system in a retracted configuration. In more detail, the first coupler member 52 is secured to proximal end 64 of positioning member 60 while the second coupler member 56 is secured to proximal end 77 of actuator member 76 and spacing member 54 is positioned between coupler members 52 & 56. The longitudinal spatial arrangement between the secured first and second coupler members 52 and 56 is such that when the coupling assembly 70 of delivery system 30 is in a retracted configuration, spacing member 54 is preferably placed in a slight state of compression. The spring force of spacing member 54 provides a bias to the maintain the coupling assembly 70 in a retracted configuration by simultaneously applying a proximally directed force to the proximal end 77 of actuator member 76 and a distally directed force to the proximal end 64 of positioning member 60. The spring force of spacing member 54 in the compressed state is sufficiently large as to overcome any tensile forces encountered during the manipulation of embolic coil 40.

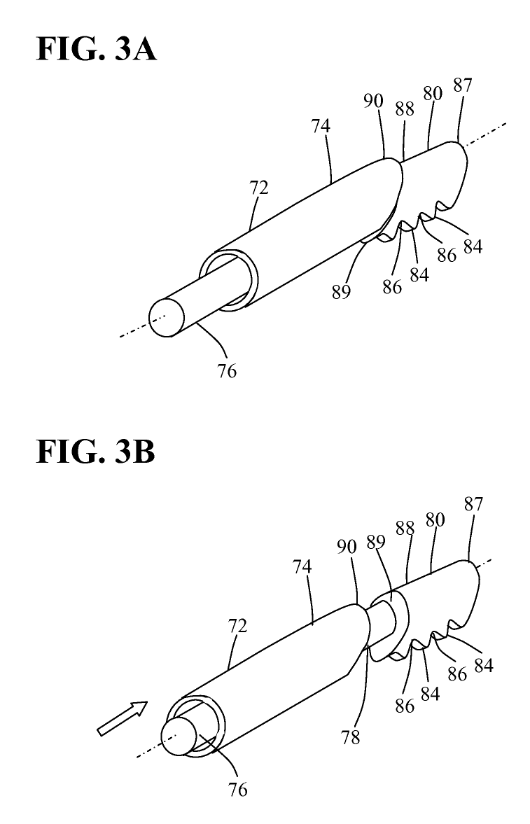

FIGS. 3A and 3B illustrate more detailed partial perspective views of coupling assembly 70 in retracted and extended configurations. As shown in FIG. 3A, actuator tip member 80 optionally includes a plurality of protrusion portions 84 and recess portions 86 positioned between distal tip 87 and proximal end 88. The protrusion and recess portions of actuator tip member 80 are dimensioned to provide better engagement with the proximal end 44 of embolic coil 40. In the retracted configuration, actuator tip member 80 proximal end 88 is positioned adjacent shaft member distal end 74 and more particularly actuator tip member proximal tip 89 is directly adjacent shaft member distal tip 90. Actuator tip member proximal tip 89 and shaft member distal tip 90 are preferably angled. FIG. 3B shows coupling assembly 70 placed in an extended configuration. Actuator assembly 50 is utilized to operatively move the coupling assembly between retracted and extended configurations. From the retracted configuration, advancing the secured second coupler member 56 towards the secured first coupler member 52, advances proximal end 77 of actuator member 76 distally relative to positioning member proximal end 64, thereby causing actuator tip member 80 to move distally relative to shaft member distal end 74. From the extended configuration, secured second coupler member 56 is moved proximally relative to secured first coupler member 52, in turn, moving proximal end 77 of actuator member 76 proximally, thereby causing actuator tip member 80 to move proximally towards shaft member distal end 74.

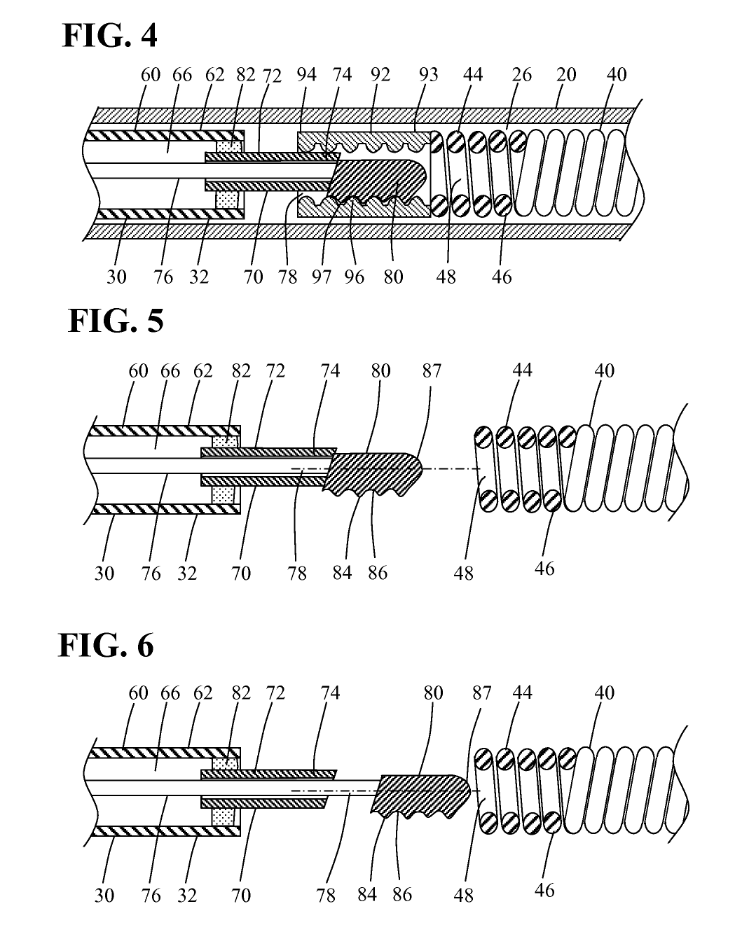

Similar to FIG. 2, FIG. 4 illustrates the construction of implant deployment system 10 with embolic coil 40 being positioned within catheter lumen 26 at catheter distal end 22 and further including a tubular headpiece 92 coupled to coil 40 and positioned at proximal end 44. Headpiece 92 has a distal end 93 and a proximal end 94 and an interior lumen 95. Located within lumen 95 of headpiece 92 are protrusion portion 96 and recess portion 97. Optionally, headpiece 92 may contain a plurality of protrusion and recess portions. Coupling assembly 70 is shown in a retracted configuration where the distal end 74 of shaft member 72 and actuator tip member 80 are positioned within lumen 95 of headpiece 92. In the retracted configuration, actuator tip member protrusion portion 84 mates with headpiece recess portion 97 while actuator tip member recess portion 86 mates with headpiece protrusion portion 96 to provide secure engagement between delivery system 30 and embolic coil 40.

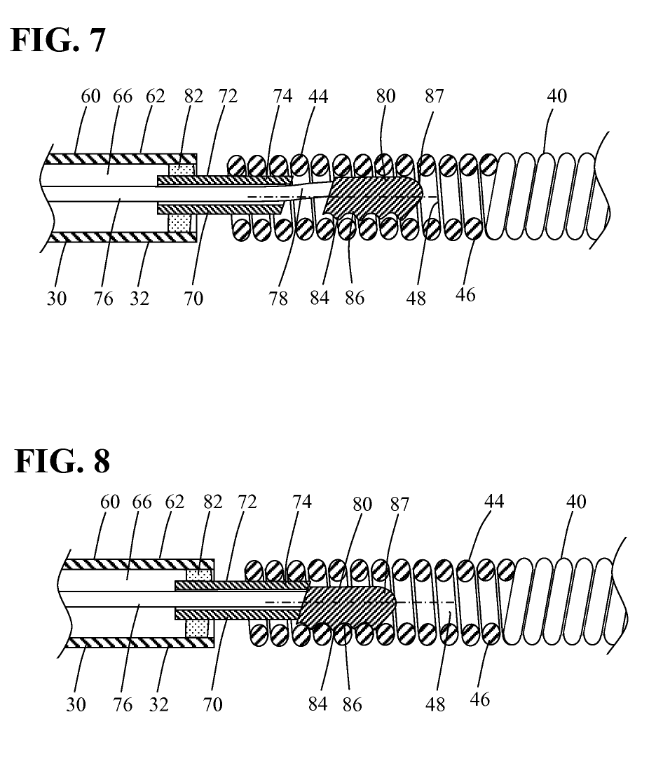

FIGS. 5 through 8 illustrate various relative positions of delivery system 30 and proximal end 44 of embolic coil 40 for clarification and discussion regarding the loading of coil 40 onto delivery system 30 or releasing coil 40 from delivery system 30. FIG. 5 depicts delivery system 30 in a retracted configuration positioned adjacent embolic coil 40. The embolic coil 40 and delivery system 30 are not engaged to show that the actuator tip member 80 has a diameter smaller than the diameter of embolic coil lumen 48 and is offset from the longitudinal axis of actuator member 76, which is coaxially positioned within the lumen of shaft member 72. The diameter of shaft member 72 at distal end 74 is also smaller than the diameter of embolic coil lumen 48. While both the diameters of shaft member distal end 74 and actuator tip member 80 are smaller than the diameter of coil lumen 48 they cannot be inserted into lumen 48 when delivery system 30 is in the retracted configuration due to the actuator tip member 80 offset. To insert actuator tip member 80 and shaft member distal end 74 into lumen 48 of embolic coil 40, delivery system 30 must operatively be placed in the extended configuration (FIG. 6) by advancing actuator member 76. As previously discussed, in the extended configuration actuator tip member 80 is positioned distal to its previous position in the retracted configuration. The distance actuator tip member 80 is moved from the retracted configuration to the extended configuration is dependent upon a number of factors including the stiffness of actuator member 76, in addition to, the dimensions of actuator tip member 80, shaft member distal end 74 and coil lumen 48. This distance typically ranges from about 0.1 mm to about 5 mm with a preferred range of about 0.2 mm to 1.0 mm. In the extended configuration, the distal end 32 of reusable delivery system 30 can be inserted into the coil lumen during the loading of a coil onto delivery system 30 or removed from the coil lumen when depositing a coil at an implantation site. FIG. 7 illustrates the distal end 32 of delivery system 30 positioned within lumen 48 of coil 40. More particularly, shaft member distal end 74 and actuator tip member 80 are positioned within coil lumen 48 at proximal end 44. To accommodate the insertion into or removal from lumen 48 of shaft member distal end 74 and actuator tip member 80, actuator member distal end 78 flexes. To complete the loading of coil 40 onto delivery system 30, the delivery system 30 is operatively placed in the retracted configuration by retracting actuator member 76 to move actuator tip member 80 proximally towards shaft member distal end 74. The proximal movement of actuator member 76, relative to shaft member 72, limits or reduces the ability of actuator member distal end 78 to flex causing actuator tip member 80 and shaft member distal end 74 cooperatively engage embolic coil 40.

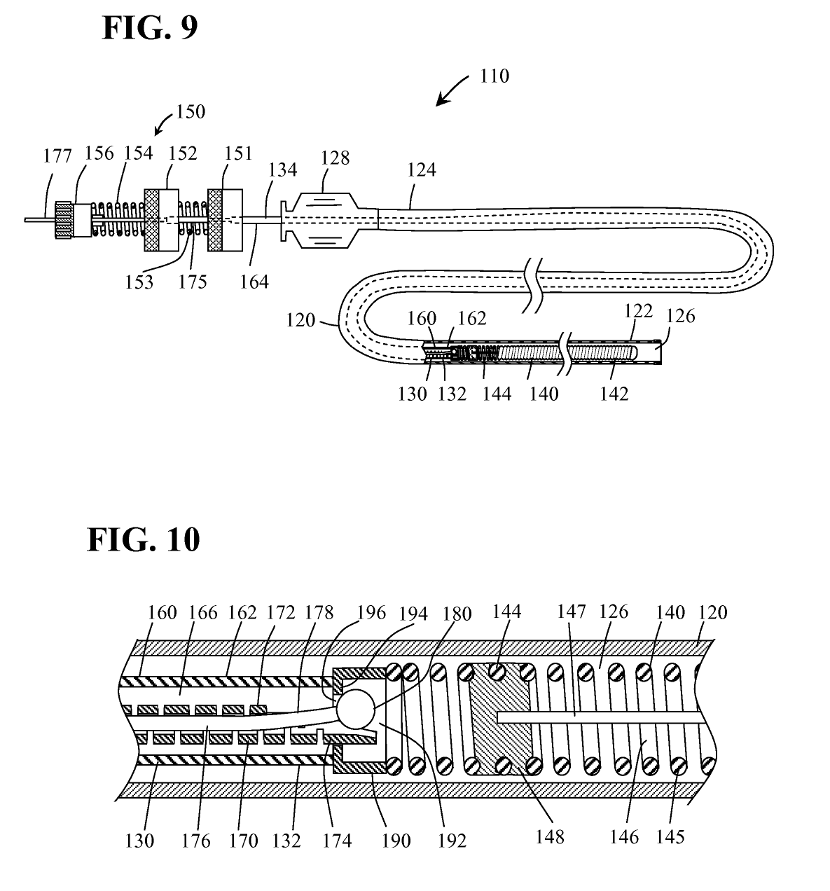

FIG. 9 generally illustrates an alternate embodiment of a medical implant deployment system 110 of the present invention which includes delivery catheter 120 having a distal end 122, a proximal end 124, a lumen 126 extending therethrough and a catheter hub 128 affixed to proximal end 124, a delivery system 130 having a distal end 132 and a proximal end 134 and an embolic coil 140 having a distal end 142 and a proximal end 144 that is releasably coupled to the distal end 132 of delivery system 130. Embolic coil 140 is a medical implant of a general type suitable for use in occluding a vessel, duct or aneurysm.

Embolic coil 140 is generally formed from a primary coil of a helically wound wire 145, made from a material which is biocompatible and preferably radio-opaque. Suitable biocompatible materials include metals such as platinum, platinum alloys, stainless steel, nitinol, tantalum and gold and plastics such as nylons, polyesters, polyolefins and fluoropolymers. The wire usually has a circular cross-section, however, non-circular cross-sections, such as “D” shapes, are used in commercially available coils. The diameter of the wire may range from about 0.0001″ to about 0.010″ and is largely dependent upon the particular clinical application for the coil. The diameter of the primary coil is generally dependent upon the wire diameter and the diameter of the mandrel used for winding. The primary coil diameter typically ranges from about 0.002″ to about 0.060″ and is also dependent upon on the clinical application. The wound primary coil is typically removed from the mandrel leaving the coil with a lumen 146. In addition to the aforementioned method of winding a coil, there are other “mandrel-less” forming processes that are suitable for making primary coils that plastically deform the wire into coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil may range from about 0.1 cm to about 150 cm with a preferred range of about 0.5 cm to about 70 cm. The distal end of the coil is typically rounded or beaded to make the coil end more atraumatic. Embolic coil 140 incorporates a stretch resistant member 147 that limits undesirable elongation of the primary coil during device manipulation. Stretch resistant member 147 is filamentous (preferably a wire having a small diameter ranging from about 0.0001″ to about 0.003″) positioned within coil lumen 146, having one end secured to coil distal end 142 (not shown) and the other end secured at coil proximal end 144 using a securing means 148. Securing means 148 preferably takes the form of solder; however other means such as welding, adhesives or mechanical interlocks may also be suitable to secure stretch resistant member 147. Other variations of embolic coils include coated or modified coils that enhance occlusion through coil surface modifications, addition of therapeutics and/or volume filling materials (foams, hydrogels, etc.).

As depicted in FIG. 9, deployment system 110 may further include an actuator assembly 150 that is positioned proximal to proximal end 124 of catheter 120. Actuator assembly 150 includes a first coupler member 151, a second coupler member 152, a first spacing member 153, a second spacing member 154 and a third coupler member 156. The first, second and third coupler members 151, 152 and 156 typically take the form of commercially available rotating hemostatic valve (RHV) like assemblies. A typical RHV-like assembly includes a housing body, a threaded cap and a compressible insert. The housing body and threaded cap are typically formed of a rigid plastic such as polystyrene, ABS, nylon or polycarbonate while the insert is formed of an elastomeric material such as silicone or rubber. The assembled housing body, cap and insert all have a contiguous aligned axial passage way. As the cap is threaded onto the housing body, the insert is compressed causing the diameter of the passageway through the insert to decrease. First spacing member 153 preferably takes the form of a spring and is positioned between first and second coupler members 151, 152. Second spacing member 154 also preferably takes the form of a spring and is positioned between second and third coupler members 152, 156. The ends of first spacing member 153 may be releasably secured (or fixedly attached) to each of the first and second coupler members 151,152. Likewise, the ends of second spacing member 154 may be releasably secured (or fixedly attached) to each of the second and third coupler members 152,156.

FIG. 10 illustrates in more detail the construction of the implant deployment system 110 with the implant, coil 140, being positioned within catheter lumen 126 at catheter distal end 122. Delivery system 130 includes a tubular positioning member 160 having a distal end 162, a proximal end 164 and a lumen 166 extending therethrough. Delivery system 130 also includes a coupling assembly 170 having a tubular shaft member 172 with a distal end 174, proximal end 175 and an elongate actuator member 176 with proximal and distal ends 177 and 178 respectively. Actuator member 176 preferably takes the form of an elongate resilient nitinol wire although other materials and forms such as tubes or cables may be suitable. Elongate actuator member 176 is positioned within the lumen of shaft member 172 and includes an actuator tip member 180 fixedly attached to actuator member distal end 178. Actuator tip member 180 preferably takes the form of a spherical bead and is generally dimensioned to be greater than the lumen diameter of shaft member 172 at distal end 174, thereby preventing proximal movement of tip member 180 into the lumen of shaft member 172 at distal end 174. Shaft member 172 is shown slidably positioned within lumen 166 with distal end 174 extending distal to positioning member distal end 162.

Additional detail shown in FIG. 10 illustrates that embolic coil 140 further includes a cap-like headpiece 190 that is positioned and secured at proximal end 144. Headpiece 190 is generally tubular and has an interior space 192 and an end wall 194 that includes aperture 196 creating a flange. As previously discussed, the proximal end 144 of embolic coil 140 is releasably coupled to the distal end 132 of delivery system 130. More particularly, the distal end 174 of shaft member 172 and the actuator tip member 180 of coupling assembly 170 are positioned within interior space 192 through aperture 196 of headpiece 190 positioned at proximal end 144 of embolic coil 140. Shown in FIG. 10, delivery system 130 is in a retracted configuration where actuator tip member 180 is positioned adjacent shaft member distal end 174 such that the diameters of shaft member distal end 174 and actuator tip member 180 cooperatively engage end wall 194 of headpiece 190 at proximal end 144 of embolic coil 140 to thereby maintain the attachment of coupling assembly 170 to embolic coil 140. The actuator assembly 150 coupled to proximal end 134 of delivery system 130, operatively maintains the delivery system in a retracted configuration. In more detail, the first coupler member 151 is secured to proximal end 164 of positioning member 160 while the second coupler member 152 (positioned proximal to first coupler member 151) is secured to proximal end 175 of shaft member 172 and first spacing member 153 is positioned between first and second coupler members 151, 152. Third coupler member 156 (positioned proximal to second coupler member 152) is secured to proximal end 177 of actuator member 176 and second spacing member 154 is positioned between coupler members 152 & 156. The longitudinal spatial arrangement between the secured second and third coupler members 152 and 156 is such that when the coupling assembly 170 of delivery system 130 is in a retracted configuration, spacing member 154 is preferably placed in a slight state of compression. The spring force of spacing member 154 provides a bias to the maintain the coupling assembly 170 in a retracted configuration by simultaneously applying a proximally directed force to the proximal end 177 of actuator member 176 and a distally directed force to the proximal end 175 of shaft member 172.

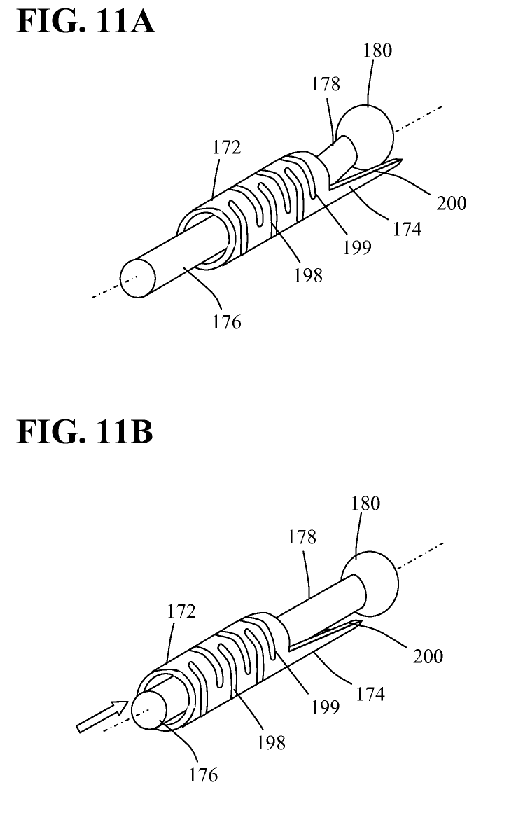

FIGS. 11A and 11B illustrate more detailed partial perspective views of coupling assembly 170 in retracted and extended configurations. As shown in FIG. 11A, actuator member 176 is coaxially positioned within the lumen of shaft member 172. Shaft member 172 preferably includes a plurality of slots 198 and 199, through the tubular wall to increase the shaft member flexibility. Preferably the plurality of slots 198 and 199 are dimensioned and arranged to increase the lateral flexibility while minimizing the longitudinal dimensional changes of shaft member 172. Shaft member 172 also includes a preferably angled, distal tip 200 located at distal end 174. Distal tip 200 is preferably formed by removing a portion of the tubular wall of shaft member 172 at distal end 174. In the retracted configuration, actuator tip member 180 is positioned on distal tip 200 such that actuator member distal end 178 is in a flexed configuration. FIG. 11B shows coupling assembly 170 placed in an extended configuration where actuator tip member 80 is positioned distal to shaft member distal end 174. Actuator assembly 150 is utilized to operatively move the coupling assembly between retracted and extended configurations. From the retracted configuration, advancing the secured third coupler member 156 towards the secured second coupler member 152, advances proximal end 177 of actuator member 176 distally relative to shaft member proximal end 175, thereby causing actuator tip member 180 to move distally relative to shaft member distal end 174. From the extended configuration, secured third coupler member 156 is moved proximally relative to secured second coupler member 152, in turn, moving proximal end 177 of actuator member 176 proximally, thereby causing actuator tip member 180 to move proximally towards shaft member distal end 174 onto the angled distal tip 200.

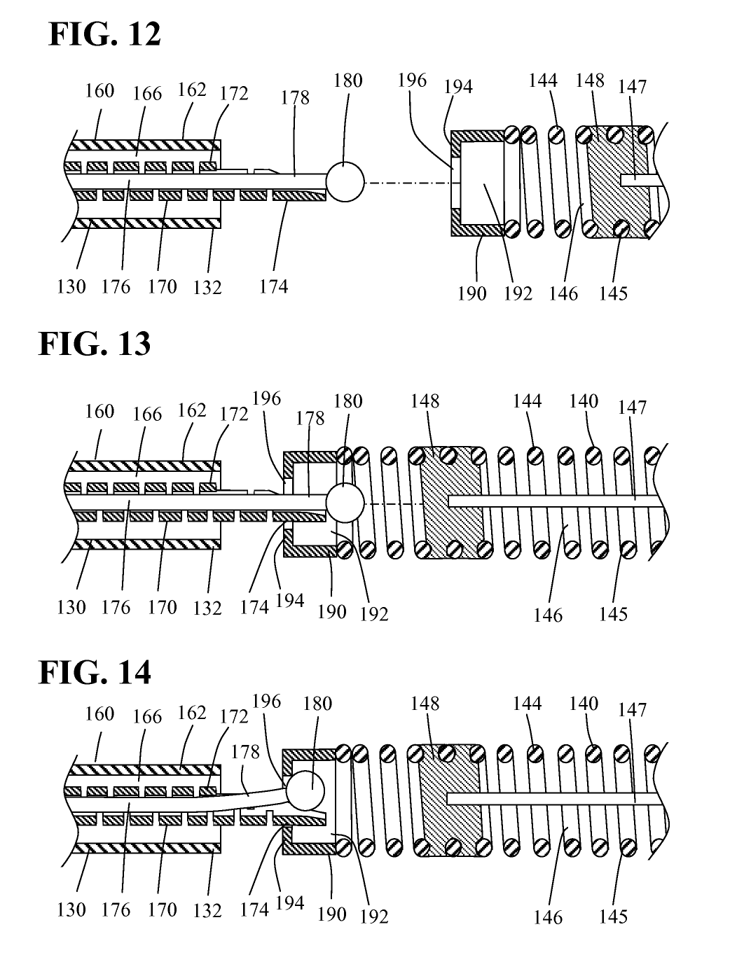

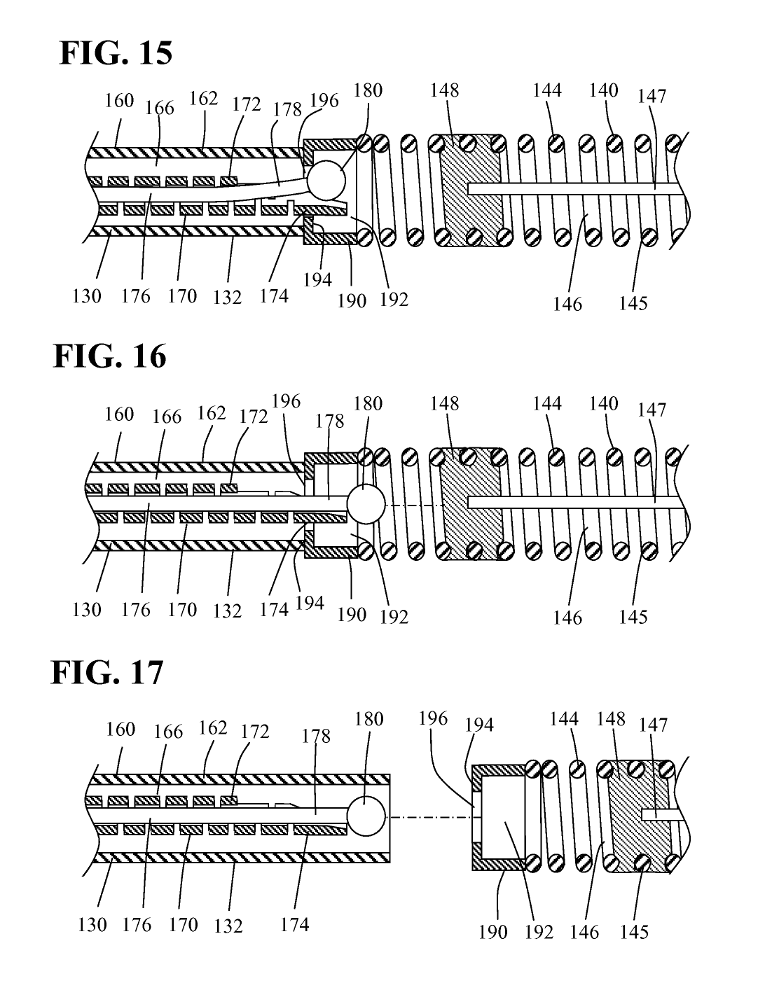

FIGS. 12 through 17 illustrate various relative positions of delivery system 130 and proximal end 144 of embolic coil 140 for clarification and discussion regarding the loading of coil 140 onto delivery system 130 or releasing coil 140 from delivery system 130. FIG. 12 depicts delivery system 130 in an extended configuration positioned adjacent embolic coil 140. The embolic coil 140 and delivery system 130 are not engaged to show that the actuator tip member 180 and shaft member distal end 174 both have diameters smaller than the diameter of aperture 196 of coil headpiece 190. While in the extended configuration, coil 140 may be loaded onto delivery system 130 by inserting actuator tip member 180 and shaft member distal end 174 of coupling assembly 170 through aperture 196 of coil headpiece 190 as shown in FIG. 13. To secure embolic coil 140 on to delivery system 130, actuator assembly 150 is operated to move coupling assembly 170 from the extended configuration to the retracted configuration in which actuator tip member 180 is positioned on shaft member distal end 174. In the retracted configuration, the relative positions of actuator tip member 180 and shaft member distal end 174 cooperate to produce an “effective diameter” which is larger than the diameter of aperture 196, thereby preventing the uncoupling of delivery system 130 from embolic coil 140 (FIG. 14). Once positioning member distal end 162 is positioned adjacent end wall 194 of headpiece 190, as shown in FIG. 15, delivery system 130 is used to delivery embolic coil 140 through a catheter to a desired implantation site in the body. Typically, distal end 162 of positioning member 160 is used to push embolic coil 140 through the catheter lumen while coupling assembly 170 is used to retract embolic coil 140, if necessary. After embolic coil 140 has been properly positioned at the implantation site and the release of the implant is desired, actuator assembly 150 is operated to place coupling assembly 170 in an extended configuration. As previously discussed, in the extended configuration actuator tip member 180 is positioned distal to its previous position in the retracted configuration. The distance actuator tip member 180 is moved from the retracted configuration to the extended configuration is dependent upon a number of factors including the stiffness of actuator member 176, in addition to, the dimensions of actuator tip member 180, shaft member distal end 174 and aperture 196. This distance typically ranges from about 0.1 mm to about 5 mm with a preferred range of about 0.2 mm to 1.0 mm. In the extended configuration, FIG. 16, actuator tip member 180 is positioned distal to shaft member distal end 174, thus removing the previous cooperative “effective diameter” that prevented uncoupling delivery system 130 from embolic coil 140. To complete the release of embolic coil 140 from delivery system 130, actuator assembly 150 is operated to retract coupling assembly 170 relative to positioning member 160, by proximally moving secured second couple member 152 relative to secured first coupler member 151, to thereby remove shaft member distal end 174 and actuator tip member 180 from aperture 196.

Numerous modifications exist that would be apparent to those having ordinary skill in the art to which this invention relates and are intended to be within the scope of the claims which follow.

That which is claimed is:

1. A vascular occlusion coil deployment system for use in placing a coil at a preselected site within a vessel or lumen comprising:
an elongated flexible positioning member having proximal and distal ends and a lumen extending therethrough;
an elongate flexible embolic coil having proximal and distal ends and a lumen at its proximal end, said embolic coil being releasably coupled to said positioning member;
a coupling assembly positioned at the distal end of said positioning member to operatively engage said embolic coil at its proximal end, said coupling assembly including a tubular shaft member having proximal and distal ends, a lumen extending therethrough and a longitudinal axis wherein said proximal end of the shaft member is fixedly coupled to the distal end of said positioning member such that the lumen of said shaft member is contiguous with the lumen of said positioning member, an elongate flexible resilient wire having proximal and distal ends and being positioned within the lumens of said positioning member and shaft member and a tip member positioned distal to said shaft member having proximal and distal portions wherein said proximal portion is fixedly coupled to the distal end of said resilient wire,
said coupling assembly having a first configuration wherein said shaft member distal end and said tip member are insertable into or removable from the lumen of said embolic coil at its proximal end and a second configuration wherein said shaft member distal end and tip member are positioned within the lumen of said coil and interlockingly engaged with said coil, said coupling assembly being movable between said first and second configurations by relative axial movement of said resilient wire.

2. A vascular occlusion coil deployment system as defined in claim 1, wherein said tip member includes protrusions adapted to engage said embolic coil when the coupling assembly is in said second configuration.

3. A vascular occlusion coil deployment system as defined in claim 1, wherein said tip member comprises an enlarged bead.

4. A vascular occlusion coil deployment system as defined in claim 1, further including an actuator assembly positioned at the proximal end of said positioning member, wherein said actuator assembly biases said coupling assembly towards said second configuration.

5. A vascular occlusion coil deployment system as defined in claim 1, wherein said shaft member distal end is angled.

6. A vascular occlusion coil deployment system as defined in claim 1, wherein said shaft member distal end includes cuts through the wall thereby increasing shaft member flexibility.

7. A vascular occlusion coil deployment system for use in placing a coil at a preselected site within a vessel or lumen comprising:
an elongate flexible embolic coil having proximal and distal ends and a lumen at its proximal end;
an elongated flexible positioning member having proximal and distal ends and a lumen extending therethrough;
a coupling assembly positioned at the distal end of said positioning member and operatively engaged with said embolic coil at its proximal end, said coupling assembly including a tubular shaft member having proximal and distal ends and a lumen extending therethrough, an elongate flexible resilient member having proximal and distal ends positioned within the lumen of said shaft member and a tip member fixedly coupled to the distal end of said resilient member,
said coupling assembly having a first configuration wherein said shaft member distal end and said resilient member are removable from the lumen of said embolic coil at its proximal end and a second configuration wherein said shaft member distal end and tip member are positioned within the lumen of said embolic coil and interlockingly engaged with said embolic coil, said coupling assembly being movable between said first and second configurations by relative axial movement between said resilient member and said shaft member.

8. A vascular occlusion coil deployment system as defined in claim 7, wherein said tip member includes protrusions adapted to engage said embolic coil when the coupling assembly is in said second configuration.

9. A vascular occlusion coil deployment system as defined in claim 7, wherein said tip member comprises an enlarged bead.

10. A vascular occlusion coil deployment system as defined in claim 7, further including an actuator assembly positioned at the proximal end of said positioning member, wherein said actuator assembly biases said coupling assembly towards said second configuration.

11. A vascular occlusion coil deployment system as defined in claim 7, wherein said shaft member distal end is angled.

12. A vascular occlusion coil deployment system as defined in claim 7, wherein said shaft member distal end includes cuts through the wall thereby increasing shaft member flexibility.

13. A medical implant deployment system for use in placing an implant at a preselected site within a vessel or lumen comprising:
a medical implant having proximal and distal ends and a coupling member having an aperture fixedly attached to the proximal end of said medical implant;
an elongated flexible positioning member having proximal and distal ends and a lumen extending therethrough;
a coupling assembly having proximal and distal ends positioned at the distal end of said positioning member and operatively engaged with the proximal end of said medical implant, said coupling assembly including a tubular shaft member having proximal and distal ends and a lumen extending therethrough, an elongate flexible resilient member having proximal and distal ends positioned within the lumen of said shaft member and a tip member fixedly coupled to the distal end of said resilient member, said coupling assembly having a first configuration wherein said tip member is positioned distal to said shaft member distal end and said coupling assembly is removable from the aperture of said coupling member and a second configuration wherein said shaft member distal end and tip member are positioned through said aperture and interlockingly engaged with said coupling member, said coupling assembly being movable from said first configuration to said second configuration by at least one of proximal movement of said resilient member relative to said shaft member and distal movement of said shaft member relative to said resilient member.

14. A medical implant deployment system as defined in claim 13, wherein said coupling member aperture includes a flange.

15. A medical implant deployment system as defined in claim 13, wherein said tip member includes protrusions adapted to engage the coupling member when the coupling assembly is in said second configuration.

16. A medical implant deployment system as defined in claim 13, wherein said tip member comprises an enlarged bead.

17. A medical implant deployment system as defined in claim 13, further including an actuator assembly positioned at the proximal end of said positioning member, wherein said actuator assembly biases said coupling assembly towards said second configuration.

18. A medical implant deployment system as defined in claim 13, wherein said shaft member distal end includes cuts through the wall thereby increasing shaft member flexibility.

19. A medical implant deployment system as defined in claim 13, wherein said coupling assembly in said second configuration has an effective diameter larger than the diameter of said aperture.
